(19) United States
(12) Patent Application Publication
(10) Pub. No.: US 2007/0293806 A1
(43) Pub. Date: Dec. 20, 2007

(54) Venous bag in an extracorporeal blood circuit

(75) Inventors: Nicola Ghelli, S. Pietro In Casale (IT); Edgardo Costa Maianti, Mirandola (IT); Roberto Balanzoni, San Giovanni Del Dosso (IT); Fabio Balugani, Cavezzo (IT)

Correspondence Address: MODIANO & ASSOCIATI, Via Meravigli, 16, MILAN, 20123, omitted

(73) Assignee: EUROSETS S.R.L.

(21) Appl. No.: 11/802,606

(22) Filed: May 24, 2007

(30) Foreign Application Priority Data
Jun. 20, 2006 (IT) MI2006A001189

(51) Int. Cl. A61B 19/00; A61M 37/00

(52) U.S. Cl. 604/6.15; 604/408

(57) ABSTRACT

A venous bag in an extracorporeal blood circuit, comprising two flat sheets which are connected one another so as to determine a closed containment volume which contains a filter, the sheets being connected at two opposite sides through concertina portions provided, at the intermediate edge, with a filament made of elastically deformable material.

### BACKGROUND OF THE INVENTION

It is known that one of the devices that are present in extracorporeal blood circuits established during certain surgical procedures is a bag which is designed to contain blood which arrives from a line connected to a vein of the patient and is also connected, for optional additions, to a container, known as cardiotomy reservoir, which collects and filters the blood that arrives from the operating field; this bag is known as “venous bag”.

Considering the presence, around the patient, of several complex devices which risk compromising or at least reducing the freedom of action of operators, it is readily evident that it is important to tend to reduce the volume of each individual device, and the aim of the present invention is to provide a venous bag which has a reduced space occupation during operation.

### SUMMARY OF THE INVENTION

This aim is achieved by a venous bag in extracorporeal blood circuit, according to the invention, comprising two flat sheets which are connected one another so as to determine a closed containment volume which contains a filter, characterized in that said sheets are connected at two opposite sides by means of concertina portions provided, at the intermediate edge, with a filament made of elastically deformable material.

### BRIEF DESCRIPTION OF THE DRAWINGS

Further characteristics and advantages of the invention will become better apparent from the description of a preferred but not exclusive embodiment thereof, illustrated by way of nonlimiting example in the accompanying drawings, wherein:

FIG. 1 is a front view of the bag in the emptying condition;

FIG. 2 is a view of the bag in the expansion condition;

FIGS. 3 and 4 are sectional views, taken respectively along the line III-III of FIG. 1 and along the line IV-IV of FIG. 2.

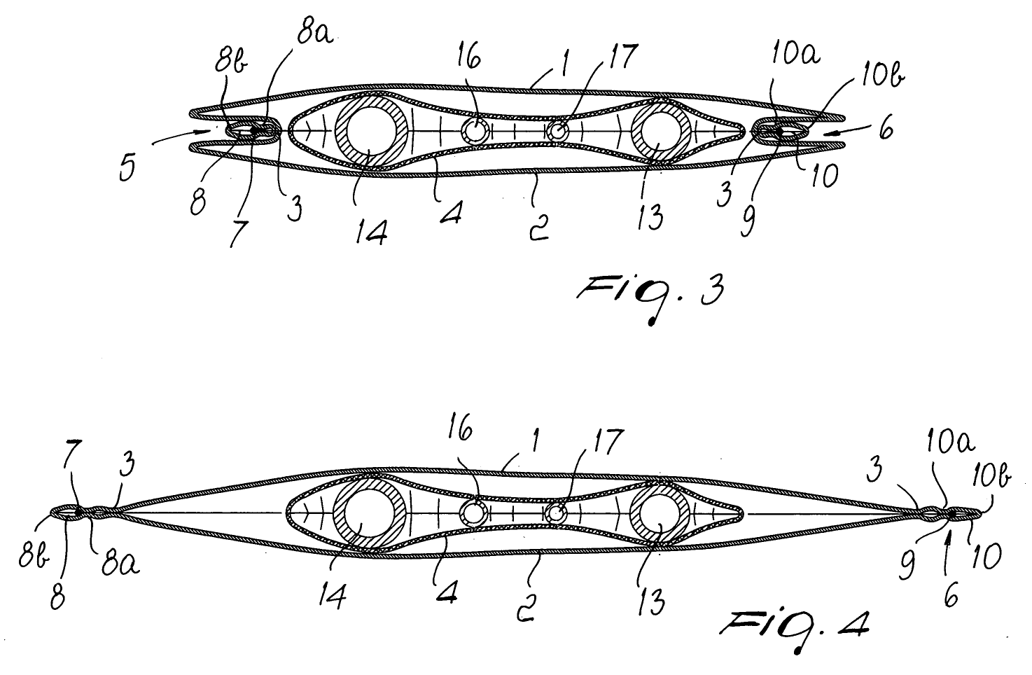

### DESCRIPTION OF THE PREFERRED EMBODIMENTS

With reference to the figures, the bag according to the invention comprises two flat plastic sheets 1 and 2, which are connected one another by means of a thermal bonding 3 so as to define a closed containment volume for containing blood, which includes a filter 4 in the form of a bag which is heat-sealed at 4a and is designed to extract any air contained in the blood.

The bag comprises two sides provided by means of concertina portions 5 and 6, each provided with a portion of filament made of elastically deformable material which is accommodated in an appropriately provided channel which extends along the intermediate edge of the concertina portion, and thus the concertina portion 5 is provided with a portion of filament 7 inserted in a channel 8 delimited by heat seals 8a, 8b and the concertina portion 6 is provided with a portion of filament 9 inserted in a channel 10 delimited by heat seals 10a, 10b.

The portions of filament 7 and 9 are joined so as to form an assembly which is monolithic with a connecting portion 11 inserted in a channel 12 at the lower head of the bag at which tubes 13 and 14 enter; such tubes are heat-sealed to the bag at 15 and are connected to lines which arrive respectively from a cardiotomy reservoir and from a vein of the patient.

The upper head of the bag comprises drains 16 and 17, and the reference numeral 18 designates a coupling which is designed to be connected to a blood outflow line.

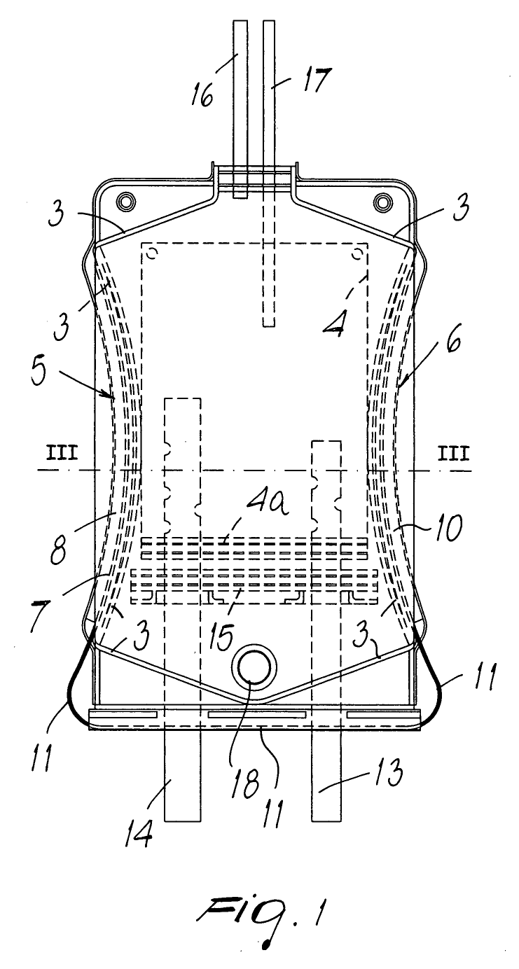

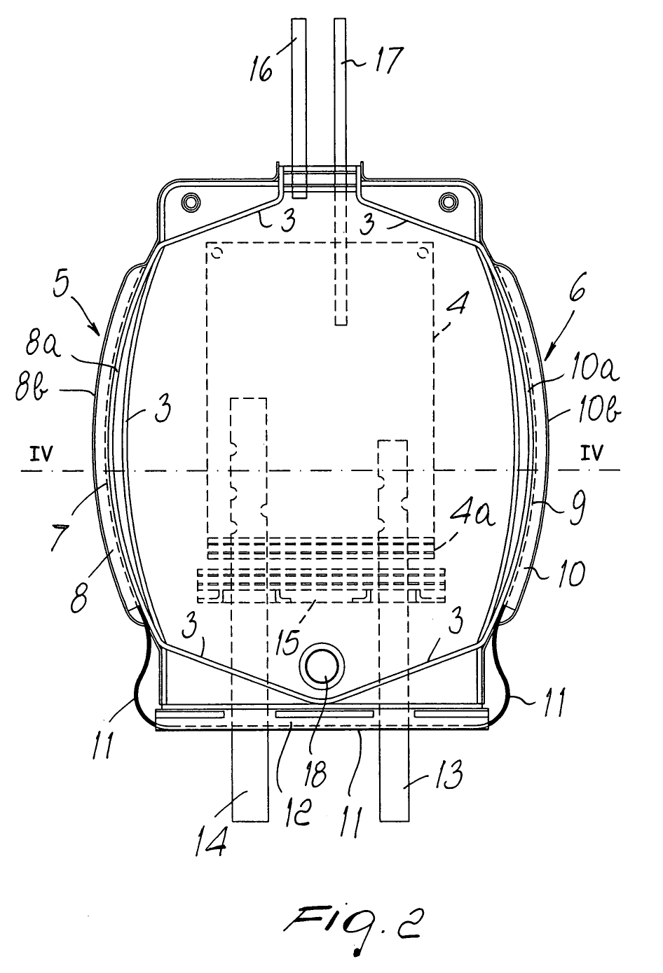

The presence of the portions of filament 7 and 9 ensures that every time emptying is performed from the expansion condition shown in FIG. 2, the bag returns to the minimum-volume condition shown in FIG. 1, and the correct operation of the filament is ensured by its monolithic configuration, which is obtained by means of the portion 11 for connecting the portions 7 and 9; it is in any case possible to ensure the correct operation of the portions of filament at the concertina portions, even if they are left independent from each other, by appropriately coupling them to the intermediate edge of the concertina portions, or by means of an external support.

The described invention is susceptible of numerous modifications and variations, all of which are within the scope of the appended claims; all the details may further be replaced with other technically equivalent elements.

The disclosures in Italian Patent Application No. MI2006A001189 from which this application claims priority are incorporated herein by reference.

What is claimed is:

1. A venous bag in an extracorporeal blood circuit, comprising two flat sheets which are connected one another so as to determine a closed containment volume which contains a filter, wherein said sheets are connected at two opposite sides by means of concertina portions provided, at an intermediate edge, with a filament made of elastically deformable material.

2. The bag according to claim 1, wherein the filament is accommodated within a channel formed at the intermediate edge of the concertina portion.

3. The bag according to claim 1, wherein portions of the filament that are present at the two concertina portions are provided continuously by means of a connecting portion which is accommodated within a channel.

4. The bag according to claim 1, wherein portions of the filament that are present at the two concertina portions are provided independently of each other.

5. The bag according to claim 1, wherein said filter is in the form of a bag, said bag comprising two sides provided by means of concertina portions, each provided with a portion of filament made of elastically deformable material which is accommodated in a channel which extends along the intermediate edge, a lower head provided with a channel which is adapted to accommodate a portion of filament for connecting the two portions provided at the concertina portions, and further provided with two blood intake tubes which are connected to lines which arrive respectively from a cardiotomy reservoir and from a patient, an upper head provided with drains, a coupling adapted to be connected to a blood outflow line being provided proximate to the lower head.
